(19) United States
(12) Patent Application Publication
(10) Pub. No.: US 2010/0279894 A1
(43) Pub. Date: Nov. 4, 2010

(54) BACTERIOPHAGES AND COATING MATERIAL FOR SURFACES

(75) Inventors: Stefanie Eiden, Leverkusen (DE); Axel Eble, Berlin (DE); Bastian Budde, Koln (DE); Sascha Plug, Leverkusen (DE); Peter Krüger, Gladbach (DE)

Correspondence Address: Briscoe, Kurt G.;Norris McLaughlin & Marcus, PA, 875 Third Avenue, 8th Floor, New York, NY, 10022, US

(73) Assignees: Bayer Technology Services GMBH, Leverkusen (DE); Bayer Material Science AG, Leverkusen (DE)

(21) Appl. No.: 12/739,015

(22) PCT Filed: Nov. 20, 2008
(86) PCT No.: PCT/EP08/09787

§ 371 Date: Apr. 21, 2010

(30) Foreign Application Priority Data
Nov. 23, 2007 (DE) 102007056818.7

(51) Int. Cl. C40B 40/02; C40B 50/06; C12N 7/00

(52) U.S. Cl. 506/14; 506/26; 435/235.1

(57) ABSTRACT

The present invention relates to a composition comprising bacteriophages and coating material for surfaces, characterized in that a first additional peptide is fused on proteins of the bacteriophage and furthermore a second additional peptide is fused on proteins of the bacteriophage. In addition, the present invention relates to a surface that has been coated with the composition and the use of the composition for coating surfaces.

The present invention relates to a composition comprising bacteriophages and coating material for surfaces. Furthermore the present invention relates to a surface that has been coated with the composition and the use of the composition for coating surfaces.

In the field of painting of surfaces there are known methods for promoting adhesion, for example bifunctional organosilane compounds, which produce a chemical bond between the polymer matrix of the paint and the inorganic substrate. A disadvantage of these adhesion promoters is, among other things, that optimal adhesion is only guaranteed if, on the surface of the substrate, there are hydroxide groups, with which the silane can interact. Therefore in many cases expensive pretreatment of the substrate is necessary. The production of made-to-order materials is also very expensive and made-to-order materials are not yet known for all substrates.

Selectively self-organizing adhesion promoters have the advantage of ensuring adhesion of paint and adhesive layers on metallic surfaces for a very long service life, as is required for example in the automobile sector for further improvement of corrosion protection. Furthermore they provide uniformly good adhesion of a uniform paint film on the composite materials with different surfaces, such as plastic/metal composites, which are finding increasing application. Moreover, the specific self-organization of biological adhesion promoters on different surfaces makes selective coating possible in simple processes, which makes new applications of composite materials possible and, also with a view to the electronics industry, offers the prospect of a simplified and precise method for the production of circuits on semiconductors.

The coupling of an inorganic substrate with biological components for modification of surface properties is known in biomimetics. Short-peptide bacteriophages selected from a phage library have previously been used for example for precipitating or separating inorganic materials (WO2003/078451). Hybrid materials consisting of an inorganic substrate and specific polypeptide ligands are also used as a potential solution for altering the substrate surface. However, identification of the biological ligand (usually a peptide) suitable for the substrate is time-consuming and expensive and so far has stood in the way of concrete application. The concept of a bifunctional ligand for binding two inorganic components has been mentioned in the prior art (see for example Sarikaya et al., Nature Materials, 2003, 2, 577-585). Concretely, the binding of cells or biomolecules on a polymer substrate, in particular on oxidized chlorine-enriched polypyrrole (PPyCl) or poly(lactate-co-glycolate) (PLGA), by bacteriophage with bifunctional binding properties was described for example in WO2004/035612. WO2004/035612 mentions the basic possibility of binding medicinal products to the substrate by this method, but exclusively describes the identification of the polymer-specific phage by so-called biopanning and binding of the phage to the polymer substrate. The further binding to another substance, which for the example of a coating material is not a biological binding partner of the phage used, is not further described, and moreover this binding would also have to take place selectively and would require precise matching of the ligand to the other binding components.

The present invention tackles the problem of providing a composition, comprising bacteriophages and coating material for surfaces, which improves the adhesion of the coating material on surfaces that are to be coated.

This problem was solved according to the invention by means of a composition comprising bacteriophages and coating material for surfaces, wherein a first additional peptide is fused to proteins of the bacteriophage and in addition a second additional peptide is fused to proteins of the bacteriophage.

Coating material for surfaces is to be understood as a layer of any material that is used as covering, protection, decoration or finish.

In the terminology used in the present invention, proteins of the bacteriophage denote gene products of the phage genome, which for example make up the coat of the bacteriophage. A first additional peptide is a peptide that is not contained on the natural form of the phage, but is presented on the phage for example by molecular-genetic manipulations of the phage genome. The peptide is fused on proteins of the bacteriophage, which means that it is joined to the protein either N-terminally or C-terminally via a peptide linkage. The first additional peptide has the property that it can form a bond to the coating material or to the surface to be coated. The type of bond can for example be a covalent bond, electrostatic interaction via charged or partially charged functional groups or a hydrogen bridge bond.

The second additional peptide is also a peptide that is not contained on the natural form of the phage, but is presented on the phage for example by molecular-genetic manipulations of the phage genome. The peptide is fused on proteins of the bacteriophage, which means that it is joined to the protein either N-terminally or C-terminally via a peptide linkage. This peptide also has the property that it can form a bond to the coating material or to the surface to be coated. The type of bond can be for example a covalent bond, electrostatic interaction via charged or partially charged functional groups or a hydrogen bridge bond.

Thus, overall, we have a bifunctional phage that makes possible or improves the adhesion of the coating material on the surface.

In one embodiment of the present invention, the first additional peptide and/or the second additional peptide have, independently of one another, a length from ≧4 amino acids to ≦100 amino acids, preferably ≧5 amino acids to ≦50 amino acids, more preferably ≧6 amino acids to ≦20 amino acids. The peptides can be linear, or also loop-shaped. Peptides with the aforementioned number of amino acids can be fused well on phage proteins by ordinary methods of molecular biology and are suitable for adhering to surfaces. Examples of such peptides are linear peptides with a length of 7 amino acids, linear peptides with a length of 12 amino acids or loop-shaped peptides with a length of 7 amino acids, which are framed by a disulfide bridge between two cysteine residues.

In another embodiment of the present invention the amino acid sequence of the first additional peptide and/or of the second additional peptide was determined by panning of a combinatorial phage population on a substrate surface. For the evolutive selection of some phage species from a large combinatorial phage population (“phage display library”) on a substrate, usually a phage display library is exposed to a substrate in a buffered aqueous environment, so that the binding of some phage can take place. Nonspecifically binding and weakly binding phages are washed away using an aqueous washing buffer. Phages that are still binding after washing and so are specific phages are then eluted using another aqueous buffer, called elution buffer hereinafter. This entire procedure is called “panning”. The eluted phages are amplified and exposed to the substrate again in further rounds of panning, until a population of phages that bind well is accumulated. Examples of this technology are given in Sarikaya et al., Nature Materials, 2003, 2, 577-585; O'Neil and Hoess, Current Opinion in Structural Biology, 1995, 5, 443-449; Smith and Scott, Methods in Enzymology 1993, 217, 228-257; Sambrook and Russell (Eds.), 2001, Molecular Cloning: A laboratory manual (third edition), Cold Spring Harbor Press, pages 18.115 to 18.122. The amino acid sequence itself, which is responsible for the binding, can be determined by methods of gene technology or molecular biology.

The selection of phage species from a combinatorial phage population can comprise the following steps:
a. a phage display library is exposed to a substrate in a buffered aqueous environment,
b. washing of the substrate,
c. elution of the binding phages using an aqueous elution buffer,
   wherein washing of the substrate can take place with input of energy by ultrasound.

Elution of the phages using ultrasound makes it possible to select and detect the phages with especially good binding. Preferably in each washing fraction the presence of phage is determined qualitatively for example by a plaque-assay spot test. It is then favorable to select the material of the substrate surface from the group comprising polymers, preferably polyethylene, polypropylene, polyurethane, polystyrene and/or polyurea; semiconductors and metals, preferably steel, iron, aluminum and/or zinc.

Advantageously, commercially available M13 phage display libraries are used, which possess a randomized peptide as gpIII-fusion protein, in particular as linear peptide with a length of 7 amino acids, as linear peptide with a length of 12 amino acids, or as loop-shaped peptide with 7 amino acids, which are framed by a disulfide bridge between two cysteine residues. A combinatorial phage display library prepared by selective alteration by genetic engineering, which possesses a randomized peptide as gpIII-fusion protein, which has a length different from 7 or 12 amino acids, is also conceivable. Alternatively, through selective alteration by genetic engineering of the phage DNA, a combinatorial phage display library can be prepared, which contains randomized peptides fused on the gpVIII protein of the M13 bacteriophage.

Preferably, the elution conditions are also adapted to the particular substrate, so that the phages that bind well are eluted completely from the surface. Elution can be carried out gently, by adding an aqueous elution buffer, with which intact phages are obtained. For this, the following parameters can be optimized:
pH value of the elution buffer usually pH 2 to pH 11.5, where the pH of the washing buffer is closer to pH 7 than the pH of the elution buffer, and preferably at least two elution steps are carried out with different elution buffers. Preferably at least one acid elution step and at least one basic elution step are carried out.
Nature and concentration of the elution buffer substance, for example alkali phosphates (usually at concentrations from 0.005 to 1.5 mol/L), Tris-HCl (0.005 to 1.5 mol/L), glycine-HCl (0.05 to 0.2 mol/L) or triethylamine (0.05 to 0.2 mol/L).
Presence, nature and concentration of salts, in particular alkali or alkaline-earth halides, sulfates, carbonates, phosphates or nitrates, preferably sodium chloride usually at concentrations from 5 to 500 mmol/L, preferably 25 to 250 mmol/L, especially preferably 50 to 200 mmol/L.
Presence, nature and concentration of detergents, preferably Tween®-20, sodium dodecylsulfate (SDS), or Triton X-100, usually at concentrations from 0.01 to 1 wt. %, preferably 0.05-0.5 wt. %.
Amount of elution buffer usually from 1 to 10 mL, temperature of the elution buffer usually 4° C. to 55° C., preferably from 15 to 40° C., elution time preferably at least 5 s.
Successive elutions can be carried out in the same or different conditions, with adjustment of the number of said elutions (e.g. one elution with 0.1 mol/l glycine pH 2.0 followed by a neutralization step with 1 mol/l Tris-HCl pH 8, followed by a second elution with 0.1 mol/l triethylamine pH 11.5 followed by a neutralization step with 1 mol/l Tris-HCl pH 7.5).

This method of the aforementioned adaptation of the washing and elution conditions to the substrate or the particle results in genetically modified phages that bind well, which can be selected in a shorter time from a combinatorial phage library than with panning procedures used hitherto.

The eluted phage are amplified and exposed once again to the substrate or the particle. If further panning rounds are carried out, preferably under more stringent washing conditions, we obtain a population of specifically binding phage, which are characterized in that they present a short peptide to their proteins that are responsible for the binding. The sequence of this peptide chain can be determined by sequencing the phage DNA. This DNA sequence can be used for genetically engineering other phage, so that they present, as fusion proteins, peptides that are capable of binding. Especially preferably, in this way bifunctional phage are produced, which bind specifically to the particle with one of the peptides, and specifically to the substrate with the other peptide.

In another embodiment of the present invention, the bacteriophages are of type M13. These phage can be varied readily and are easily obtainable, and can be multiplied easily in culture. As gene products, they have for example the proteins gpIII, gpV, gpVI, gpVII, gpVIII and gpIX. It is possible for the first additional peptide to be fused to the gpIII or gpVIII protein and the second additional peptide to be fused to the gpIII or gpVIII protein. Lengthening of these proteins with additional peptides, i.e. fusing, can be carried out readily on these proteins. It is especially preferable if the gpVIII protein is fused N-terminally with a peptide of the sequence STTRLR. Additionally, the gpIII protein can also be fused N-terminally with a peptide of the sequence ADKSAHVSLISR, so that a bifunctional phage is formed. The first-mentioned sequence enables the phage to bind to polyurethane surfaces. The latter sequence enables it to bind to steel surfaces.

A system according to the present invention can be constructed with respect to the phage so that on a phage of type M13 preselected by panning with a first additional peptide already fused to gpIII, which imparts binding properties to a surface, a second additional peptide is fused to the gpVIII protein of the same phage clone, wherein the peptides fused to gpIII and gpVIII can be identical or different.

Moreover, a system according to the present invention is also conceivable, wherein on a phage of type M13 preselected by panning with a first additional peptide already fused to gpIII, a second additional peptide is fused to the gpVIII protein of the same phage clone, which imparts binding properties to a surface, wherein the peptides fused to gpIII and gpVIII can be identical or different.

Such phage can be prepared by a method that comprises the following steps:
a. restriction sites are inserted into the gpVIII procoat sequence, by carrying out site-specific mutagenesis on the gpVIII procoat gene, which is located on a plasmid (preferably pUC) between two suitable restriction sites (preferably PagI and KpnI),
b. the mutations from step a) are transformed by recloning from the plasmid to the replicative form (RF) of the M13 genome, preferably in the form of the M13KE vector,
c. the replicative form of the phage DNA is cut with the restriction enzymes whose sites were inserted by the mutations from step a), and then serves for taking up suitable complementary oligonucleotides, which display, at the ends, the corresponding overhang for ligating into the open restriction sites, and which code for a second additional peptide sequence.

Preferably, through the panning and selection process there is already a peptide fused to gpIII, which imparts binding properties. In order to achieve bifunctionality, usually another peptide, which also imparts a binding property, is fused to the gpVIII protein of the same phage clone through molecular-biological manipulation of the phage genome, wherein the peptides fused to gpIII and gpVIII can be identical or different, so that multiple binding properties can be achieved. In order to achieve this fusion, restriction sites are first inserted in the corresponding position of the phage genome. For cloning purposes, the mutations are first introduced on another plasmid, preferably pUC, which contains the gpVIII procoat gene between two suitable restriction sites (preferably PagI and KpnI). Insertion of the new restriction sites into the gpVIII procoat sequence takes place by site-specific mutagenesis (e.g. Sambrook and Russell (Eds.), 2001, Molecular Cloning: A laboratory manual (third edition), Cold Spring Harbor Press, pages 8.42 to 8.45) by using oligonucleotides with suitable sequences, for example:

Mutagenic Oligonucleotides

| | |
|---|---|
| Nco-A | 5′-CGGCGTTCCCATGGTGTCTTTCGCTGC-3′ |
| Nco-B | 5′-GCAGCGAAAGACACCATGGGAACGCCG-3′ |
| Pst-A | 5′-GCTGTCTTTCGCTGCAGAGGGTGACGATCCC-3′ |
| Pst-B | 5′-GGGATCGTCACCCTCTGCAGCGAAAGACAGC-3′ |

The inserted restriction sites can be selected from the group comprising: Aar I, Aat II, Acc I, Acu I, Afl II, Age I, Ahd I, Apa I, ApaL I, Asc I, AsiS I, Avr II, BamH I, Bbs I, Bcg I, BciV I, Bcl I, BfrB I, Blp I, BmgB I, Bmt I, Bsa I, Bsg I, BsiW I, BspE I, BssH II, BssS I, BstAP I, BstB I, BstE II, BstX I, BstZ17 I, CspC I, EcoN I, EcoR V, Fse I, FspA I, Hinc II, Hpa I, I-Ceu I, I-Sce I, Mfe I, Mlu I, Nco I, Nhe I, Not I, Nru I, Nsi I, PI-Psp I, PI-Sce I, PaeR7 I, PflF I, PflM I, Pme I, Pml I, PshA I, PspOM I, Pst I, Rsr II, Sac I, Sac II, Sal I, SanD I, Sap I, Sca I, SexA I, Sfi I, SgrA I, Sma I, Spe I, Srf I, Stu I, Sty I, Tli I, Tth111 I, Xba I, X cm I, Xho I, Xma I, Zra I. Preferably an NcoI site is inserted in the signal sequence of gpVIII and a PstI site in the mature region. These mutations are then transformed by recloning from the plasmid into the replicative form (RF) of the M13 genome, preferably in the form of the M13KE vector (New England Biolabs). For this, the vector is cut off with suitable restriction enzymes, preferably PagI and KpnI, and the fragment obtained is ligated into the also cut M13KE vector. For the execution of restriction and ligation steps with double-stranded plasmids see e.g. Sambrook and Russell (Eds.), 2001, Molecular Cloning: A laboratory manual (third edition), Cold Spring Harbor Press, pages 1.84 to 1.87. For the execution of transformations of plasmids in E. coli bacteria see Sambrook and Russell (Eds.), 2001, Molecular Cloning: A laboratory manual (third edition), Cold Spring Harbor Press, pages 1.105-1.122.

After inserting the sites in the M13 genome, the replicative form of the phage DNA can usually be cut up with the two restriction enzymes NcoI and PstI and then serves for taking up suitable complementary oligonucleotides, which display, at the ends, the corresponding overhang for ligation into the open restriction sites, and which code for an adhesion-promoting peptide sequence. The information for this adhesion-promoting peptide sequence can be derived from the panning procedure of the gpIII phage display library on a suitable substrate. Through ligation of the DNA and transformation into suitable host bacteria, bifunctional phage can be produced, which optionally present, both on their gpIII and on their gpVIII proteins, different adhesion-promoting peptide sequences.

Alternatively, instead of oligonucleotides that code for an adhesion-promoting peptide sequence, it is also possible for library oligonucleotides to be ligated in the suitably cut M13 genome. These are usually two complementary oligonucleotides, which display, at the ends, the corresponding overhang for ligating into the open restriction sites. In addition, they have a region of usually 15 bases, which are freely variable. By ligation of the hybridized oligonucleotides into the cut RF-DNA and transformation into suitable host bacteria, we obtain a randomized gpVIII phage library. The phage can then be selected on substrate surfaces by the panning procedures already described.

Advantageously, the coating material for surfaces is selected from the group comprising adhesion promoters, primers, paint and/or adhesive.

Adhesion promoters are bifunctional, low-molecular compounds, one functionality of which brings about adhesion on the surface that is to be coated or glued, whereas the other functionality is responsible for anchoring in the paint or adhesive film. Organosiloxanes or organosilanes are commonly used as adhesion promoters, which for example promote adhesion on glass surfaces, and on metal surfaces. Derivatives of complexing agents represent another type of adhesion promoters. The complex-forming group increases the adhesion to a metallic surface, whereas a functional group leads to anchoring in the paint or adhesive film. Derivatives of hydroquinoline thus increase adhesion to aluminum surfaces, and derivatives of gallic acid increase adhesion to steel surfaces.

Primers are for example polymer-bound films on the faying part that is to be coated or glued, which are similar to the adhesive or paint raw material and contain other components, for example for corrosion protection or for improved adhesion. Coats of primer are provided to improve adhesion and protect the surface against corrosion. Known examples include monuron-accelerated epoxy resins as corrosion-inhibiting priming coats. By using priming coats, in certain circumstances pretreatment for protecting the surface (plasma treatment of plastic surfaces, phosphatizing of metals) can be omitted.

Paints are coating materials that are applied on objects and form, at least partially, a continuous film. Possible constituents of a paint are: film-forming resin, hardener, solvents, water, additives, pigments, antifoaming agents, light stabilizers, optical brighteners, corrosion inhibitors, antioxidants, algicides, plasticizers, flow control agents and wetting agents. As film-forming resins, it is possible to use the polymer compounds that are known by a person skilled in the art of coating technology, such as polyesters, polyethers, polyurethanes, polyacrylates and/or polyureas, which after application on a surface result in a continuous paint film.

Preferably, film-forming resins are used that have functional groups that are available for chemical crosslinking. Such groups can be free or reversibly blocked hydroxy, thio, amino, epoxy and/or isocyanate groups. Examples of reversibly-blocked isocyanate groups are those that are blocked with acetone oxime, butanone oxime, ε-caprolactam, 3,5-dimethylpyrazole, dimethylmalonate or phenol. The blocking reagent is only cleaved at elevated temperatures, with release of the isocyanate group. This leads to higher thermal stability.

It is especially preferable for the film-forming resin to be a mixture of OH- and/or NH-functional binders, preferably polyols and/or polyamines and hardeners, preferably polyisocyanates, blocked polyisocyanates, and/or epoxy resins.

Both the resins and the hardeners can be both in solvent-free form and in the form of a solution in organic solvents or an aqueous dispersion.

Preferably the molar ratio of the functional groups of resin available for crosslinking and optionally blocked hardener is ≧1.5 to ≦2.

Another object of the present invention relates to a surface that has been coated with a composition according to the present invention. In an advantageous embodiment the material of the surface is selected from the group comprising polymers, polyethylene, polypropylene, polyurethane, polystyrene, polyurea, semiconductors, metals, steel, iron, aluminum and/or zinc.

Another object of the present invention is the use of the composition according to the invention for the coating of surfaces.

The present invention will be explained below with Examples 1 and 2.

### EXAMPLES

### Example 1

### Panning of a Combinatorial Phage Display Library (Ph.D.-12™, New England Biolabs) on a Polyurethane Substrate

Polyurethane substrate was produced from a mixture of equivalent amounts of Desmophen® 670 BA and Desmodur® N3300 (Bayer Material Science AG), with curing for 16 h at room temperature. The substrate, 20 mg, was equilibrated for 10 min in Tris-buffered saline (TBS, consisting of 50 mmol/l Tris-HCl pH 7.5, 150 mmol/l NaCl) and incubated for 60 min with 4*10¹⁰ pfu (10 μl of the original library) in 1 ml TBS at room temperature. The substrate was washed ten times with 10 ml TBST each time (TBS plus 0.1 vol. % Tween-20) (by brief vortexing, five-minute rotation plus 5 s ultrasonic bath). The first elution was carried out in acid conditions by immersing the substrate in 1 ml of 0.1 mol/l glycine pH 2.5 for 10 s followed by neutralization of the substrate in 1 ml of 0.1 mol/l Tris-HCl pH 8 for 1 min The first elution solution was neutralized by adding 200 μl of 1 mol/l Tris-HCl pH 8. The second elution was carried out in basic conditions by immersing the substrate in 1 ml of 0.1 mol/l triethylamine pH 11.5 for 1 min followed by neutralization of the substrate in 1 ml of 0.1 mol/l Tris-HCl pH 7.5 for 1 min The second elution solution was neutralized by adding 200 μl of 1 mol/l Tris-HCl pH 7.5. The substrate was then stored in TBST, for observing temporal elution effects of uneluted phage.

In each fraction, the presence of phage could now be determined qualitatively by a plaque-assay spot test (see FIG. 1). For this, bacteria of the strain E. coli ER2738 (New England Biolabs) were plated on an LB-Tet agar plate (15 g/l agar, 10 g/l Bacto-Tryptone, 5 g/l yeast extract, 5 g/l NaCl, 20 mg/l tetracycline) and incubated overnight at 37° C. 10 ml of LB-Tet medium (10 g/l Bacto-Tryptone, 5 g/l yeast extract, 5 g/l NaCl, 20 mg/l tetracycline) were inoculated with a single colony of ER2738 and shaken at 37° C. until the OD₆₀₀ was 0.4. From this, 400 μl was pipetted into 3 ml of molten LB-Agar-Top (7 g/l agar, 10 g/l Bacto-Tryptone, 5 g/l yeast extract, 5 g/l NaCl) and poured onto an LB-IPTG/X-gal plate (15 g/l agar, 10 g/l Bacto-Tryptone, 5 g/l yeast extract, 5 g/l NaCl, 1.25 mg/l isopropyl-β-D-thiogalactopyranoside, 1 mg/l of 5-bromo-4-chloro-3-indoxyl-β-D-galactopyranoside). After hardening, 3 μl of each fraction was dropped onto the plate and the latter was incubated overnight at 37° C. The presence of phage in each fraction was indicated by blue plaques.

The elution fractions (elution 1 and 2 and the two neutralizing solutions) were combined and amplified. For this, 10 ml of LB-Tet medium with 100 μl of an overnight culture of ER2738 (in LB-Tet) and the combined elution fractions were inoculated and shaken for 4.5 h at 37° C. The culture was centrifuged for 10 min at 4500×g and 4° C., and the supernatant was then centrifuged again. The upper ⅘ vol. of the supernatant was combined with ⅕ vol. of PEG/NaCl (20% (w/v) polyethylene glycol-8000, 2.5 mol/l NaCl) and incubated for 1 h at 4° C. The phage were removed by centrifugation for 20 min at 16000×g and 4° C. and resuspended in 1 ml TBS (50 mM Tris-HCl pH 7.5, 150 mM NaCl). The solution is centrifuged for 5 min at 10000×g and 4° C., 200 μl PEG/NaCl is added to the supernatant and it is incubated on ice for 60 min The phage were removed by centrifugation for 10 min at 14000×g and 4° C. and resuspended in 200 μl TBS.

The titer of the phage solution was determined by a plaque-assay. For this, 10 ml of LB-Tet medium was inoculated with a single colony of ER2738 and shaken at 37° C. until OD₆₀₀ was 0.4. From this, 400 μl was pipetted into 3 ml of molten LB-Agar-Top, 10 μl of an appropriate dilution of the phage solution was added and it was distributed on an LB-IPTG/X-gal plate. After hardening, the plate was incubated overnight at 37° C. The titer of the phage solution (in pfu/ml) could be calculated from the number of blue plaques.

In the next rounds, the complete procedure for panning was repeated, but using 1-2×10¹¹ pfu of the amplified eluate as a new library, and the Tween-20 concentration is increased to 0.5% (v/v).

A total of three panning rounds were carried out. In the resultant population there was enrichment of specifically polyurethane-binding phage clones, whose identity could be determined by sequencing the variable region of their genome. For this, individual phage clones, e.g. from the LB-IPTG/X-gal plate of the titration of the eluate of the third round, were picked out with an inoculating loop and amplified separately: 2 ml of LB-Tet medium was inoculated with 100 μl of an overnight culture of ER2738 (in LB-Tet) and the phage clone, and shaken at 37° C. for 4.5 h. The culture was centrifuged for 10 min at 4500×g and 4° C., and the supernatant was then centrifuged again. PEG/NaCl solution, 500 μl, was added to 1 ml of the phage solution and incubated for 2 h at 4° C. The phage were removed by centrifugation for 15 min at 14000×g and 4° C. and resuspended in 100 μl of 10 mM Tris-HCl pH 8.0, 1 mM EDTA, 4 M Nat. The DNA was precipitated with 250 μl ethanol for 10 min and centrifuged for 15 min at 14000×g and 20° C. The pellet was washed with 70% ethanol, centrifuged for 1 min at 14000×g and 20° C., dried and resuspended in 30 μl of 10 mM Tris-HCl pH 8.0. DNA sequencing was carried out with the primer 5′-CCCTCATAGTTAGCGTAACG-3′, obtaining the anticodon strand of the M13 DNA.

### Example 2

### Production of a Combinatorial gpVIII Phage Display Library

To insert an oligonucleotide bank for the VIII gene (main coat protein) in the M13 genome, three primers are required:

1. Library Oligonucleotide

5′ CGTCACCCTCTGCAGC(NNN)₆AGCAGCGAAAGACACCATGGGAAGC

2. Extension Primer I

5′ CGTTCCCATGGTGTCTTTC

3. Extension Primer II

5′ ATCGTCACCCTCTGCA

First a PCR reaction is carried out on the library oligonucleotide with both extension primers, for preparing the library. The library is then digested with NcoI and PstI and inserted into the corresponding restriction sites in a M13KE vector provided with NcoI and PstI sites.

So as to be able to insert the library in the M13 genome, restriction sites are first inserted into the corresponding position of the genome of M13KE. For cloning purposes, the mutations are first introduced on another plasmid (pUC), which contains the gpVIII procoat gene between the restriction sites PagI and KpnI (pUC-gp8). Insertion of the new restriction sites in the gpVIII procoat sequence takes place by site-specific mutagenesis (e.g. Sambrook and Russell (Eds.), 2001, Molecular Cloning: A laboratory manual (third edition), Cold Spring Harbor Press, pages 8.42 to 8.45) by using the following oligonucleotides:

Mutagenic Oligonucleotides

| | |
|---|---|
| Nco-A | 5′-CGGCGTTCCCATGGTGTCTTTCGCTGC-3′ |
| Nco-B | 5′-GCAGCGAAAGACACCATGGGAACGCCG-3′ |
| Pst-A | 5′-GCTGTCTTTCGCTGCAGAGGGTGACGATCCC-3′ |
| Pst-B | 5′-GGGATCGTCACCCTCTGCAGCGAAAGACAGC-3′ |

As a result an NcoI site is inserted in the signal sequence of gpVIII and a PstI site in the mature region (pUC-gp8 mut). These mutations are then transformed by recloning from pUC-gp8mut into the replicative form (RF) of the M13KE genome. For this, pUC-gp8mut is cut off with PagI and KpnI and the fragment obtained is ligated into the M13KE vector, which has also been cut. For the execution of restriction and ligation steps with double-stranded plasmids, see e.g. Sambrook and Russell (Eds.), 2001, Molecular Cloning: A laboratory manual (third edition), Cold Spring Harbor Press, pages 1.84 to 1.87. For the execution of transformations of plasmids in E. coli bacteria see Sambrook and Russell (Eds.), 2001, Molecular Cloning: A laboratory manual (third edition), Cold Spring Harbor Press, pages 1.105-1.122.

Following insertion of the sites in the genome of M13KE (M13KE-gp8 mut), its RF-DNA is cut off with the two restriction enzymes NcoI and PstI and the similarly cut library oligonucleotide is ligated into this vector.

Each ligation product is different in the 6 NNN positions and therefore codes for other amino acids at these 6 positions. The ligation preparations represent the combinatorial gpVIII library and can be used directly for panning on a substrate, similarly to a commercial gpIII phage display library.

### Example 3

### Production of a Bifunctional Bacteriophage, which Possesses a Peptide with Length of 12 Amino Acids, Fused to gpIII, and Binding Specifically to Steel, and a Peptide with Length of 6 Amino Acids, Fused to gpVIII, and Binding Specifically to Polyurethane

By panning a commercial phage library (Ph.-D.-12™, New England Biolabs) on steel, we had, in a phage clone (“St101-13”) a peptide (ADKSAHVSLISR), with length of 12 amino acids, fused to gpIII, which possessed steel binding properties. In order to achieve bifunctionality, another peptide (STTRLR), with length of 6 amino acids, which possessed polyurethane binding properties, was fused to the gpVIII protein of the same phage clone. The clone with this sequence (“PUR01-1”) was found by panning a randomized gpVIII phage display library (see above) on polyurethane.

To achieve combination, first the replicative form of PUR01-1 was cut with the restriction enzymes PagI and KpnI. The insert containing the gpVIII gene with fused polyurethane-binding peptide was ligated into the replicative form of St101-13, also cut with PagI and KpnI. By transformation in competent E. coli ER2738 bacteria (New England Biolabs), bifunctional phage were produced, which present different adhesion-promoting peptide sequences on their gpIII proteins and on their gpVIII proteins.

### DRAWINGS

FIG. 1: Plaque assay of panning fractions (round 1), dropped undiluted on an LB-Agar-Top with added E. coli ER2738 on an LB-IPTG/X-gal plate.

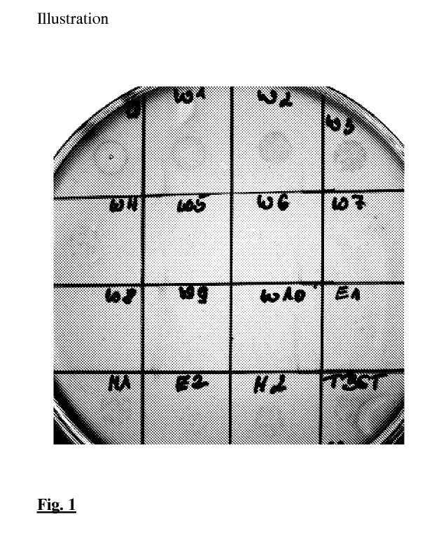

The labels on the fields denote the following:

0: Library from round 1 after substrate incubation;
W1-10: Washing fractions 1-10 (ultrasound used);
E1: Elution 1 (acidic);

### N1: Neutralization 1;

E2: Elution 2 (basic);

### N2: Neutralization 2;

TBST: Storing of the piece of substrate after panning in TBST.

1. A composition comprising bacteriophages and coating material for surfaces, wherein a first additional peptide is fused on proteins of the bacteriophage and in that in addition a second additional peptide is fused on proteins of the bacteriophage.

2. The composition as claimed in claim 1, wherein the first additional peptide and/or the second additional peptide, independently of one another, have a length from ≧4 amino acids to ≦100 amino acids.

3. The composition as claimed in claim 1, wherein the amino acid sequence of the first additional peptide and/or of the second additional peptide was determined by panning a combinatorial phage population on a substrate surface.

4. The composition as claimed in claim 3, wherein the material of the substrate surface is selected from the group consisting of polymers, semiconductors and metals.

5. The composition as claimed in claim 1, wherein the bacteriophages are of type M13.

6. The composition as claimed in claim 5, wherein the first additional peptide is fused to the gpIII or gpVIII protein and the second additional peptide is fused to the gpIII or gpVIII protein.

7. The composition as claimed in claim 6, wherein the gpVIII protein is fused N-terminally with a peptide of sequence STTRLR and additionally the gpIII protein is fused N-terminally with a peptide of sequence ADKSAHVSLISR, so that a bifunctional phage is formed.

8. The composition as claimed in claim 1, wherein the coating material for surfaces is selected from the group consisting of adhesion promoters, primers, paints and adhesives.

9. The composition as claimed in claim 8, wherein the coating material for surfaces comprises a film-forming resin, selected from the group consisting of polyesters, polyethers, polyurethanes, polyacrylates and polyureas.

10. The composition as claimed in claim 9, wherein the film-forming resin has functional groups available for chemical crosslinking.

11. The composition as claimed in claim 10, wherein the film-forming resin represents a mixture of OH- and/or NH-functional binders.

12. A surface coated with a composition as claimed in claim 1.

13. The surface as claimed in claim 12, which is composed of a material selected from the group consisting of polymers, polyethylene, polypropylene, polyurethane, polystyrene, polyurea, semiconductors, metals, steel, iron, aluminum and zinc.

14. A method of coating a surface, said method comprising coating the surface with the composition as claimed in claim 1.


SEQUENCE LISTING (7 sequences)

SEQ ID NO: 1 - DNA, 27 bp, Artificial
  primer-bind
cggcgttcccatggtgtctttcgctgc

SEQ ID NO: 2 - DNA, 27 bp, Artificial
  primer-bind
gcagcgaaagacaccatgggaacgccg

SEQ ID NO: 3 - DNA, 31 bp, Artificial
  primer-bind
gctgtctttcgctgcagagggtgacgatccc

SEQ ID NO: 4 - DNA, 31 bp, Artificial
  primer-bind
gggatcgtcaccctctgcagcgaaagacagc

SEQ ID NO: 5 - DNA, 59 bp, Artificial
  primer-bind
cgtcaccctctgcagcnnnnnnnnnnnnnnnnnnagcagcgaaagacaccatgggaagc

SEQ ID NO: 6 - DNA, 19 bp, Artificial
  primer-bind
cgttcccatggtgtctttc

SEQ ID NO: 7 - DNA, 16 bp, Artificial
  primer-bind
atcgtcaccctctgca